(19) United States
(12) Patent Application Publication
(10) Pub. No.: US 2008/0234685 A1
(43) Pub. Date: Sep. 25, 2008

(54) Patient-Adapted Osteotomy Template for Precise Resection of the Neck of the Femur in a Total Hip Prosthesis Operation

(75) Inventor: Jan Magne Gjerde, Trondheim (NO)

Correspondence Address: CHRISTIAN D. ABEL, ONSAGERS AS, POSTBOKS 6963 ST. OLAVS PLASS, NORWAY, N-0130, NO

(73) Assignee: SCANDINAVIAN CUSTOMIZED PROSTHESIS ASA, Trondheim (NO)

(21) Appl. No.: 11/568,982

(22) PCT Filed: May 13, 2005
(86) PCT No.: PCT/NO05/00164

§ 371 Date: Feb. 5, 2007

(30) Foreign Application Priority Data
May 18, 2004 (NO) 20042041

(51) Int. Cl. A61B 17/15

(52) U.S. Cl. 606/89

(57) ABSTRACT

A patient-adapted osteotomy template for precise resection of the femur in a total hip prosthesis operation with the use of a reamer comprises an upper abutment surface for abutment with the femur, a connecting surface fixed to the upper abutment surface and a guide surface, a guide slot provided in the connecting surface and an orientation indicator fixed to or provided in the osteotomy template. The guide surface and slot define the desired cutting plane and provide guidance of the resection instrument for the resection operation when the upper abutment surface is in abutment against the femur and when the osteotomy template is rotated about the drilling axis of the reamer so that the orientation indicator is in a specific position.

The invention relates to a patient-adapted osteotomy template (resection guide) for precise resection of the neck of the femur in a total hip prosthesis operation.

The traditional technique employed in operation of a total hip prosthesis involves making an opening into the hip joint with a skin incision of approximately 25 cm. This opening entails a substantial trauma for the patient with an incision through muscles and ligaments as well as blood loss. The normal length of hospitalization after a traditional hip operation is 7-11 days.

The use of minimal invasive technique involves a skin incision of approximately 7 cm. This reduces the trauma to the patient, with less pain, less blood loss, shorter convalescence, faster stabilisation and exercising of the joint. This will also give the prosthesis a better fixation and function, which in turn will contribute to an increased lifetime for the prosthesis. By means of minimal invasive technique it will be possible to perform a total hip operation with considerably shorter hospitalization time and at best as outpatients' surgery, which will also be important for the economy of the hospitals.

Hip operations are performed by the head of the femur being cut off and replaced by a femoral component that is fixed down in the femoral canal, a ball that is mounted on the femoral component, together with a cup that is fixed in the pelvis. A critical factor in the minimal invasive technique is to have a clear overall view of the wound during the operation in order to achieve precise surgery and the correct positioning for the prosthetic components. A patient-adapted prosthesis with a high degree of pre-operative planning will increase reliability and precision during a hip operation with minimal invasive technique to give a good post-operative result.

In the precise surgery associated with the implantation of a prosthesis, it is extremely important for the head of the femur to be removed with great precision in relation to the planned level. For this purpose an osteotomy template is required whereby the level and orientation of the planned cutting plane is transferred to the patient's femur with great precision. With patient-adapted prostheses, a computer model of the patient's femur will often be produced, based on CT images and the prosthesis is designed on the basis of this model. Based on this model the optimal cutting plane for the patient's femur can also be determined.

Osteotomy templates exist for use in conventional surgery, but in connection with minimal invasive technique there will be a need for osteotomy templates with entirely different characteristics. A resection guide for use in minimal invasive technique will differ greatly from those used in conventional surgery as it has to be considerably smaller, be intended for insertion through different openings, employ different anatomical landmarks and be easier to position in the planned position than in conventional surgery.

U.S. Pat. No. 5,578,037 and WO 0226145 illustrate examples of osteotomy templates for use in conventional hip surgery. These instruments are large and not suitable for use in minimal invasive technique. They are also intended for use in several patients, and therefore when used in the individual patient their precision will not be optimal. They are composed of several parts that have to be dismantled for cleaning between each operation and then correctly assembled before the next operation in order to give the correct result, thus leaving room for error. The use of such osteotomy templates also exposes them to substantial mechanical stresses, with the result that they have to be regularly checked and calibrated with respect to function and precision. For these reasons conventional osteotomy templates are expensive to produce and use, in addition to which they make greater demands on and entail more work for the surgeon.

Since traditional osteotomy templates are not adapted to the individual patient, they require the patients to have relatively similar anatomical characteristics. This means that for patients with anatomical deviations, there is a reduction in precision with traditional multi-use osteotomy templates, thereby giving a final result that is not optimal. For patients with greater anatomical deviations, moreover, the majority of multi-use templates will be difficult to use on account of limitations in the possibilities for adjustment. Even for patients without great deviations, when using multi-use templates it will be necessary to carry out adjustments of the osteotomy template during the operation, resulting in an increased risk of error, increased operating time and thereby an increased risk of complications.

There is therefore a need for an osteotomy template for use in hip operations with the use of a reamer, which provides a high degree of precision in the resection for patients with all types of hip complaints, is cheap and easy to produce and easy to use.

The object of the invention is to provide an osteotomy template that meets this requirement.

The object of the invention is achieved by means of an osteotomy template according to the patent claims.

The osteotomy template according to the invention comprises an upper abutment surface for abutment against the femur, a connecting surface fixed to the upper abutment surface and a guide surface, a guide slot provided in the connecting surface and an orientation indicator fixed to or mounted in the osteotomy template, where the guide surface and slot define the desired cutting plane and provide guidance of the resection instrument for the resection when the upper abutment surface is in abutment with the femur and when the osteotomy template is rotated about the drilling axis of the reamer, with the result that the orientation indicator is in a specific position.

The osteotomy template is preferably patient-adapted, an osteotomy template being provided for each patient that is adapted in size and shape to the patient's femur and the cutting plane being determined for each osteotomy template on the basis of the individual patient's femur. By this means greater precision is obtained in the resection for each patient.

The adaptation of size and shape is undertaken on the basis of a 3-dimensional model which, for example, may be provided by means of CT images.

The upper abutment surface is intended to provide correct positioning of the osteotomy template in the longitudinal direction of the femur and may, for example, be adapted for abutment against the trochanter major or caput. In an embodiment the abutment against the femur is implemented by the abutment surface being located in a plane that is substantially perpendicular to the drilling axis of the reamer. This may be particularly expedient in an embodiment where the abutment surface abuts against the trochanter major. Alternatively, the abutment surface may be located in a plane that forms an angle with the drilling axis of the reamer.

The connecting surface constitutes a connection between the upper abutment surface and the guide surface and/or a support for the guide slot that provides guidance of the resection instrument, for example a saw. The connecting surface may be designed in size and shape according to the patient's anatomy.

The guide surface may-be arranged on the side of the connecting surface facing the femur. In this case the guide surface or one or its edges may form an abutment portion against the femur. Alternatively, the guide surface may be arranged on the side of the connecting surface facing away from the femur. The guide surface may be arranged on one of the connecting surface's surfaces or at one of its edges. The guide surface will form an angle with the connecting surface and support the resection tool during the resection. The guide slot provides further support/guidance of the resection tool, with the result that the guide surface and the guide slot together ensure that the resection is carried out in the desired cutting plane. The angles of the guide surface and the guide slot unambiguously determine the angle of the cutting plane relative to the drilling axis.

In an embodiment the guide slot is provided at the transition between the connecting surface and the guide surface. However, the guide slot may be provided at any desirable location in the connecting surface.

While the abutment surface provides longitudinal positioning, the orientation indicator provides rotational positioning.

In an embodiment the orientation indicator is an abutment portion adapted for abutment against the femur, the appointed position of the orientation indicator being achieved when the abutment portion is in abutment against the femur, for example at the calcar. The abutment portion may be composed of a separate part connected to the connecting surface, or it may be a part of the connecting surface or another suitable portion of the osteotomy template. In an embodiment the abutment portion is composed of one of the guide surface's edges. The abutment portion may comprise a section/edge adapted to the part of the femur against which is has to abut, or it may be composed of a straight edge or be of another suitable shape.

In another embodiment the orientation indicator is a marking on the upper abutment surface and the appointed position is achieved when the marking is parallel with the patient's knee kondyle plane or with the horizontal plane. In a further embodiment the orientation indicator is a marking or edge on the osteotomy template and the appointed position is achieved when the marking or edge forms a given angle with the knee kondyle plane or horizontal plane. Other types of orientation indicators may also be envisaged.

The adaptation of the size and shape of the osteotomy template may, for example, be implemented by the distance between the upper abutment surface and the abutment portion being determined on the basis of the distance between the trochanter major/caput and the part of the femur against which the abutment surface has to abut in the individual patient. In an embodiment the abutment portion is adapted for abutment against the calcar, and the distance between the upper abutment surface and the abutment portion is then determined by the distance between the trochanter major and the calcar.

Other measurements that can be determined on the basis of the patient's anatomy are the angle between the guide slot and the upper abutment surface, which determines the desired resection angle. This angle is determined in the planning phase in order to conserve as much as possible of the patient's original bone, while retaining the possibility of implanting the prosthetic component with its design into the desired position in the femur.

The osteotomy template may also comprise a support surface that is substantially parallel to the upper abutment surface, and where both surfaces have holes adapted for the insertion of a reamer. The object of the support surface is to ensure that the abutment surface is at the correct angle relative to the reamer. The holes are arranged along a line parallel to the reamer's drilling axis, thus locating the osteotomy template at a fixed angle to the drilling axis when it is mounted on a reamer. In this state the osteotomy template can be rotated about the drilling axis, and the angular position is determined as described above by means of the orientation indicator. The support surface may be connected to the upper abutment surface or be connected to the osteotomy template in another fashion. Alternatively, the osteotomy template comprises a second support device, such as for example a piece of tube adapted for insertion of a reamer.

In an embodiment the osteotomy template is made of one piece of material, for example a single steel plate that is cut and folded into the desired shape. Alternatively, the individual parts may be welded together or the part may be constructed by means of other suitable methods, preferably in such a manner that the osteotomy template is composed of a single piece. An advantage that can be achieved by manufacturing the osteotomy template by cutting and folding a single plate is that smooth transitions are obtained between the individual parts, thus simplifying cleaning.

Since the osteotomy template can be made of one piece of material, it is not necessary to assemble it before use, thus simplifying the use and eliminating errors due to incorrect assembly. Nor is there any risk of the template's individual parts working loose and changing their relative positions during resection, which can happen with traditional assembled templates.

In an embodiment the invention also comprises a method for manufacturing a patient-adapted osteotomy template for guiding a resection instrument along a desired cutting plane for a femur in a total hip prosthesis operation with the use of a reamer, where the osteotomy template is adapted to be inserted in the reamer, where the method comprises the following steps:
providing images of the femur that has to be resected,
on the basis of the images of the femur:
to determine the position of the optimal cutting plane,
to determine the position of an upper abutment surface adapted for abutment with the upper part of the femur,
to provide the upper abutment surface and a connecting surface connected with the abutment surface and a guide surface with a guide slot, where, based on the images of the femur, the extent of the connecting surface is selected so that the plane defined by the guide'surface and the guide slot coincide with the optimal cutting plane,
to provide an orientation indicator for indicating correct rotation of the osteotomy template about the drilling axis of the reamer.

In an embodiment of the invention the orientation indicator is an abutment portion adapted for abutment against the femur, and correct rotation is achieved by rotating the osteotomy template so that the abutment portion comes into abutment against the femur.

In a further embodiment the distance between the upper abutment surface and the abutment portion is determined on the basis of the distance between the trochanter major and the calcar in the individual patient.

The invention will now be described in more detail by means of an example with reference to the appended figures.

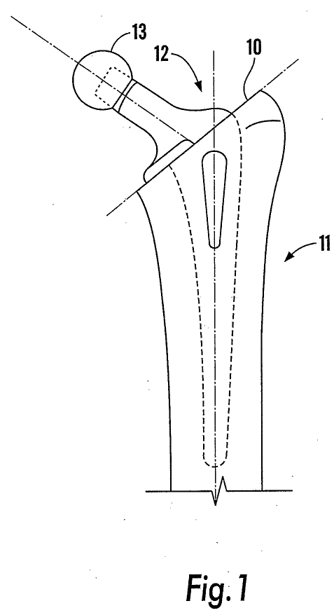

FIG. 1 is an illustration of a femur with a prosthesis.

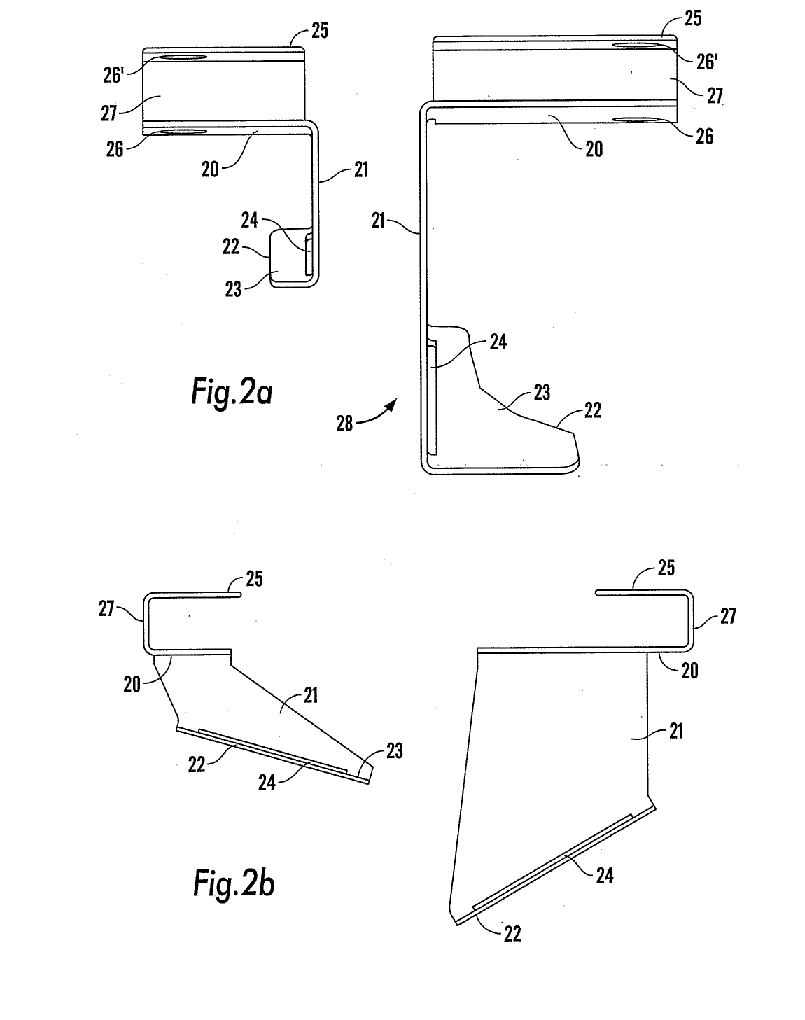

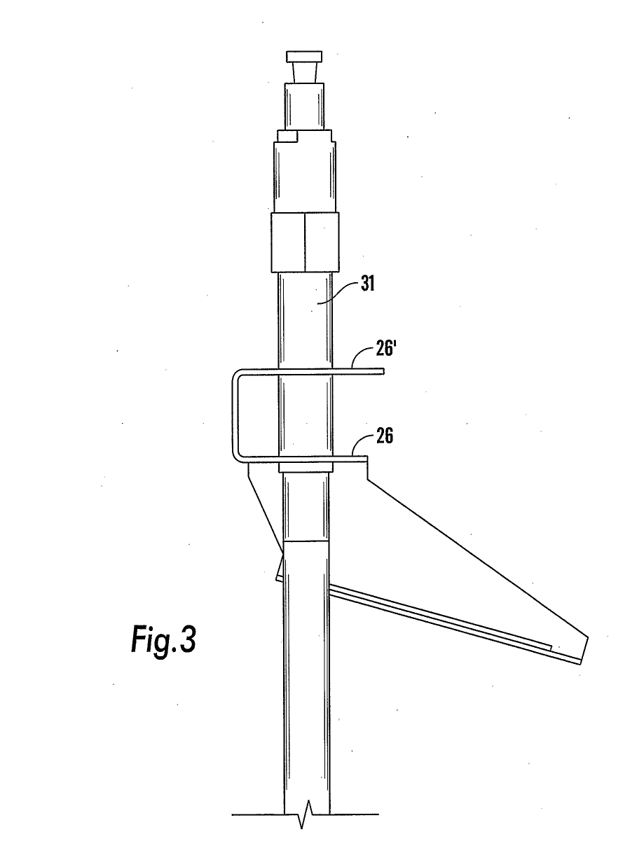

FIG. 2a illustrates two osteotomy templates according to an embodiment of the invention viewed from in front.

FIG. 2b illustrates the same two osteotomy templates as in FIG. 2a viewed from the side.

FIG. 3 illustrates the embodiment in FIGS. 2a and 2b assembled with a reamer.

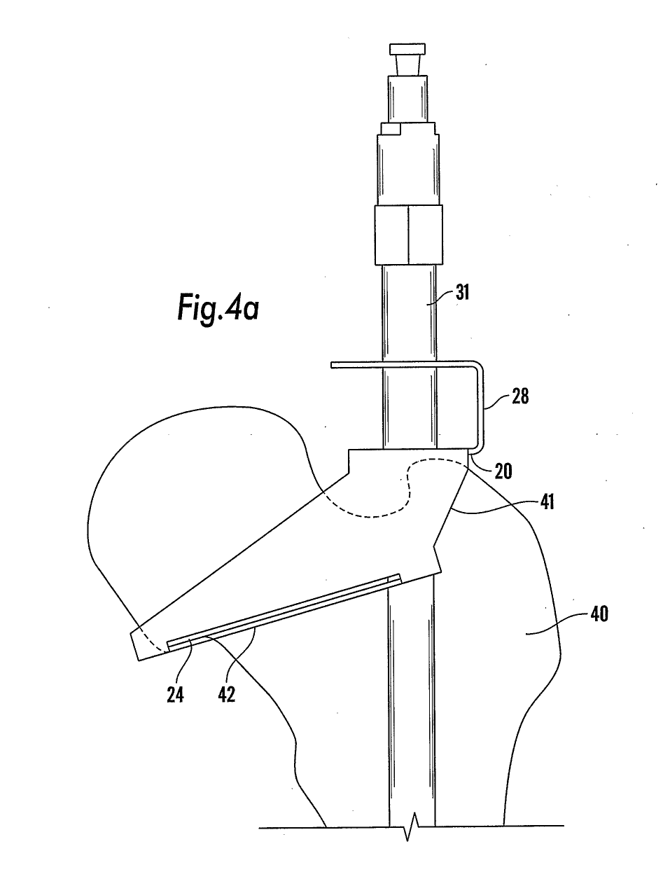

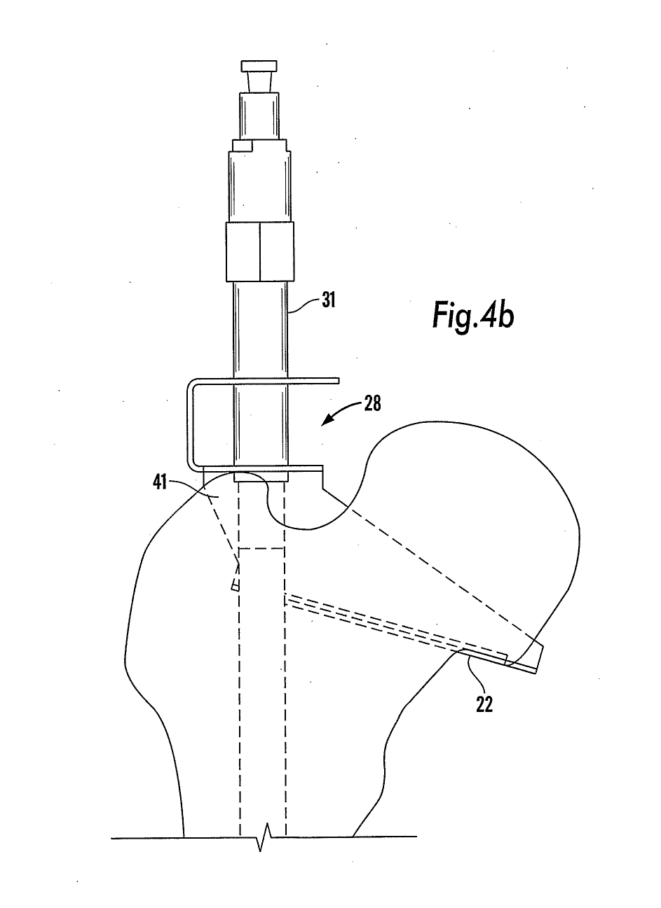

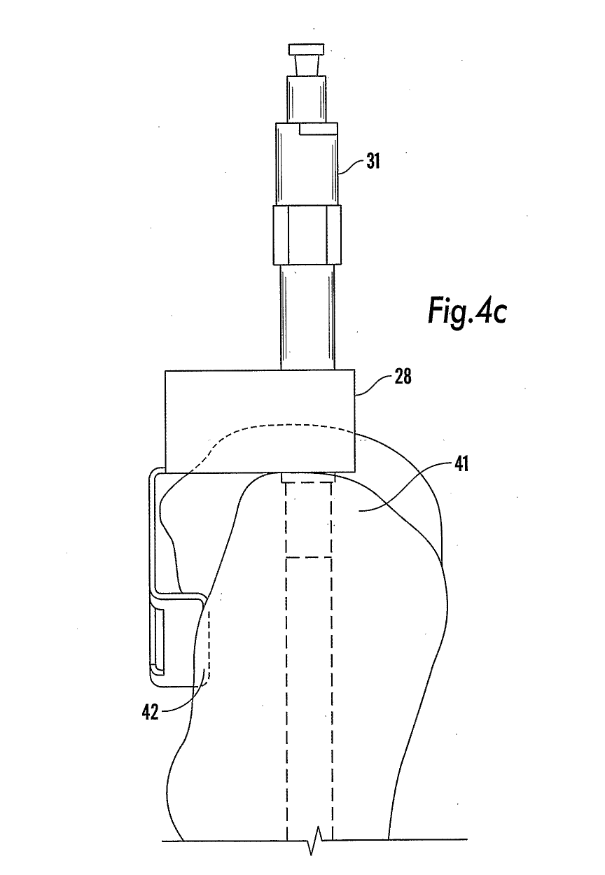

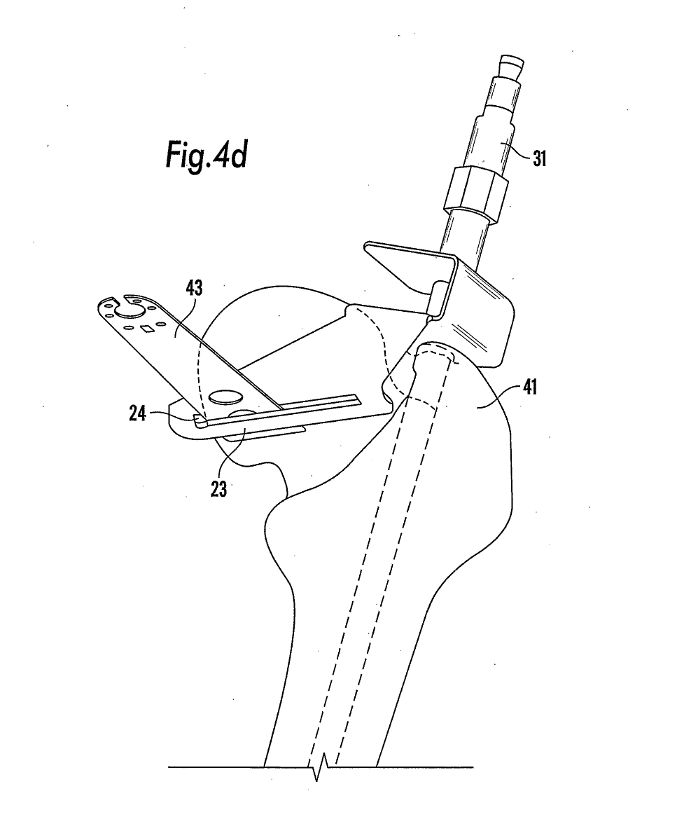

FIGS. 4a to 4d illustrate the embodiment in FIGS. 1-3 placed on a femur for use.

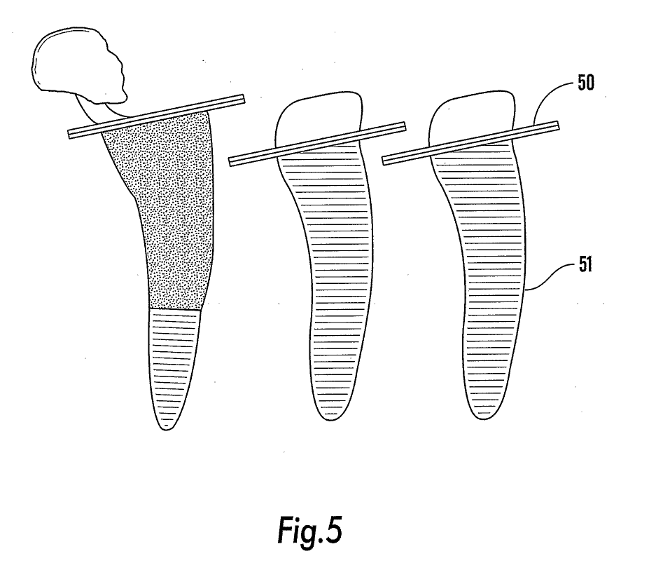

FIG. 5 illustrates how a cutting plane can be planned by means of computer models of the femur.

FIG. 1 illustrates a femur 11 where the femoral head (caput) is removed along a cutting plane 10. A prosthesis 13 is implanted in the femur along a hole drilled along a drilling axis 12.

FIG. 2a illustrates an osteotomy template 28 according to an embodiment of the invention that can be used for resection of a femur as illustrated in FIG. 1. The figure shows two examples of the same embodiment in different sizes, i.e. adapted to suit two different patients. The osteotomy template 28 comprises an upper abutment surface 20, a connecting surface 21, a guide surface 23, an orientation indicator composed of an abutment portion 22, which in this case is one of the guide surface's edges, and a guide slot 24, which is disposed in the transition between the connecting surface 21 and the guide surface 23. This embodiment also comprises a support plate 25 mounted directly over the upper abutment surface and connected with it by a transition plate 27. Two circular holes 26 and 26′ are also shown, arranged directly above each other in the two surfaces. The two holes are adapted to the size of a reamer 31, thus enabling the drill to be inserted in the holes during use. In this manner the angle relative to the drilling axis is fixed, and the osteotomy template 28 is only free to rotate about the drilling axis.

FIG. 2b illustrates the osteotomy templates depicted in FIG. 2a viewed from the side.

FIG. 3 illustrates the smallest osteotomy template from FIG. 2 mounted on a reamer as described above.

FIGS. 4a to 4d illustrate how the osteotomy template 28 in the embodiment described above abuts against a femur 40 during use. In FIG. 4b the reamer 31 is inserted along the desired drilling axis and the osteotomy template 28 is inserted into the reamer 31 until the upper abutment surface 20 comes into abutment against the trochanter major 41 on the femur. The osteotomy template 28 is then free to rotate about the drilling axis until the abutment portion 22 comes into abutment against the caput 42 as illustrated in FIG. 4c. When the osteotomy template abuts in this manner, the cutting plane is defined by the guide surface 23 and the guide slot 24. The resection is preformed by guiding a saw 43 or the like along the guide slot 24 with abutment against the guide surface 23 as illustrated in FIG. 4d. Correct resection is thereby guaranteed.

FIG. 5 illustrates computer models 51 of the femur and how the optimal cutting plane 50 can be determined on the basis of such models. The computer models may, for example, be provided by means of CT images. The optimal cutting plane is selected by comparing the prosthesis that is to be used with the model of the femur, thereby finding where the resection has to be undertaken in order to achieve the best result.

1. A patient-adapted osteotomy template for guiding a resection instrument along a desired cutting plane for a femur in a total hip prosthesis operation with the use of a reamer, where the osteotomy template is adapted to be inserted in the reamer, characterised in that it comprises:
an upper abutment surface for abutment with the femur,
a connecting surface fixed to:
the upper abutment surface and
a guide surface,
a guide slot arranged in the connecting surface and
an orientation indicator fixed to or mounted in the osteotomy template, where the guide surface and slot define the desired cutting plane and provide guidance of the resection instrument for the resection when the upper abutment surface is in abutment against the femur and when the osteotomy template is rotated about the drilling axis of the reamer so that the orientation indicator has a specific position, where the osteotomy template is composed of a single piece.

2. A patient-adapted osteotomy template according to claim 1, characterised in that the orientation indicator is an abutment portion adapted for abutment against the femur, the appointed position being achieved when the abutment portion is in abutment against the femur.

3. A patient-adapted osteotomy template according to claim 1, characterised in that the orientation indicator is a marking on the upper abutment surface, the appointed position being achieved when the marking is parallel to the patient's knee kondyle plane or to the horizontal plane.

4. A patient-adapted osteotomy template according to claim 1, characterised in that the orientation indicator is composed of a marking or an edge on the osteotomy template, the appointed position being achieved when the marking or the edge forms a given angle with the knee kondyle plane or the horizontal plane.

5. A patient-adapted osteotomy template according to claim 1, characterised in that the abutment surface is adapted for abutment against the trochanter major in a plane substantially perpendicular to the drilling axis of the reamer.

6. A patient-adapted osteotomy template according to claim 1, characterised in that the abutment surface is adapted for abutment against the caput.

7. A patient-adapted osteotomy template according to claim 2, characterised in that the abutment surface is composed of one of the guide surface's edges, and that the guide slot is arranged at the transition between the connecting surface and the guide surface.

8. A patient-adapted osteotomy template according to claim 2, characterised in that the abutment surface is adapted for abutment against the calcar.

9. A patient-adapted osteotomy template according to claim 1, characterised in that it comprises an additional surface that is substantially parallel to the upper abutment surface, where the two parallel surfaces have holes disposed along a line parallel to the drilling axis of a reamer.

10. A patient-adapted osteotomy template according to claim 1, characterised in that the osteotomy template is made of a single piece of material.

11. A patient-adapted osteotomy template according to claim 1, characterised in that the osteotomy template is made of a plate that is cut and folded in order to provide the desired shape.

12. A patient-adapted osteotomy template according to claim 1, characterised in that the distance between the upper abutment surface and the abutment portion is determined on the basis of the distance between the trochanter major and the calcar in the individual patient.

13. A method for manufacturing a patient-adapted osteotomy template for guiding a resection instrument along a desired cutting plane for a femur in a total hip prosthesis operation with the use of a reamer, where the osteotomy template is adapted to be inserted in the reamer, characterised in that the method comprises the following steps:
providing images of the femur that has to be resected,
on the basis of the images of the femur:
to determine the position of the optimal cutting plane,
to determine the position of an upper abutment surface adapted for abutment with the upper part of the femur,
to provide the upper abutment surface and a connecting surface connected with the abutment surface and a guide surface with a guide slot, where, based on the images of the femur, the extent of the connecting surface is selected so that the plane defined by the guide surface and the guide slot coincide with the optimal cutting plane,
to provide an orientation indicator for indicating correct rotation of the osteotomy template about the drilling axis of the reamer.

14. A method according to claim 13,
characterised in that the orientation indicator is an abutment portion adapted for abutment against the femur, and that correct rotation is achieved by rotating the osteotomy template so that the abutment portion comes into abutment against the femur.

15. A method according to claim 13,
characterised in that the distance between the upper abutment surface and the abutment portion is determined on the basis of the distance between the trochanter major and the calcar in the individual patient.
